(19) United States
(12) Patent Application Publication
(10) Pub. No.: US 2010/0041165 A1
(43) Pub. Date: Feb. 18, 2010

(54) PROBE-IMMOBILIZED CARRIER STORING MANUFACTURING CONDITION DATA AND MANUFACTURING METHOD AND APPARATUS THEREOF, DETECTING METHOD OF TARGET SUBSTANCE BY USE OF THE PROBE-IMMOBILIZED CARRIER, AND MEASURING APPARATUS, RECORDING MEDIUM, KIT AND SYSTEM FOR USE IN THE DETECTING METHOD

(75) Inventors: Tohru Ishibashi, Tokyo (JP); Kazuaki Takahata, Mitaka-shi (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 11/993,704

(22) PCT Filed: Jun. 29, 2006
(86) PCT No.: PCT/JP2006/313448

§ 371 Date: Dec. 21, 2007

(30) Foreign Application Priority Data
Jun. 30, 2005 (JP) 2005-191697

(51) Int. Cl. G01N 33/53; G01N 33/566; C23C 16/52; B05C 11/00; C12N 15/00; C12Q 1/68; G01N 37/00

(52) U.S. Cl. 436/501; 427/8; 118/712; 422/68.1; 422/61

(57) ABSTRACT

A target substance is more accurately detected by recording a manufacturing condition specific to a probe-immobilized carrier that influences a measurement result for the detection of the target substance, and correcting the measurement result obtained from the detection of the target substance by use of the probe-immobilized carrier on the basis of the recorded manufacturing condition. The influence of variations in the immobilization states of the probe onto the solid phase carrier on the measurement result of the target substance can be eliminated.

### TECHNICAL FIELD

The present invention relates to a probe-immobilized carrier storing manufacturing condition data and a manufacturing method and apparatus thereof, a detecting method of a target substance by use of the probe-immobilized carrier, and a measuring apparatus, recording medium, kit and network utilization system for use in the detecting method.

### BACKGROUND ART

One example of a probe-immobilized carrier includes DNA chips. The DNA chip is a high-density array in which a large number of DNA fragments or oligonucleotides quite effective as probes for the simultaneous analysis of gene expression, mutation, polymorphism, and so on are aligned on the surface of a solid phase. For example, U.S. Pat. No. 5,688,642 has disclosed a solid-phase oligonucleotide array produced by use of photolithography. The pamphlet of International Publication No. WO 95/25116 and U.S. Pat. No. 5,688,642 have disclosed a producing method of a solid-phase DNA probe array by use of an ink-jet method. At the step of detection treatment of target substances, so-called hybridization reaction is generally performed, wherein the target substances are labeled with a fluorescent material or the like and contacted with probes of the solid-phase probe array to form hybrids. In general, this hybridization reaction is a reaction through which the solid-phase probe array is contacted with or immersed in a solution of the target substances dissolved therein and then heated. Target substance concentrations and reaction temperatures in the hybridization differ depending on the combination of probes and target substances. The presence or absence of binding between the probes and the target substances is further measured with an apparatus such as fluorescence detectors.

### DISCLOSURE OF THE INVENTION

Probes used in these DNA chips can be classified broadly into those using cDNA and oligonucleotide. In both cases, the probes are expensive or valuable and are desired to be used in the minimum amount. To respond to such demands, the use of a probe-immobilized carrier having a solid phase carrier immobilizing a trace amount of probes thereon has been studied in various ways. When probes are utilized in an amount as small as possible, the acquisition of accurate measurement results requires strictly measuring the binding force (bound amount) of a target substance to the probe. For this purpose, each probe is desired to be bound in accurately the same shape and amount with the solid phase carrier. However, for handling a trace amount of probes, it is sometimes difficult to equalize the shapes of areas immobilizing probes thereon, the density of the immobilized probes, etc., which influence measurement results, for each probe or each probe-immobilized carrier. Particularly, gene diagnosis for disease, the detection of microorganisms as pathogens, and so on require precise measurement with higher accuracy. Thus, a method that can eliminate the influence of variations in the immobilization states of probes on measurement results is important.

An object of the present invention is to provide a detecting method of a target substance that can eliminate the influence of variations in the immobilization states of probes of a probe-immobilized carrier onto the solid phase carrier on a measurement result of the target substance, and to provide the probe-immobilized carrier and a measuring apparatus for use in the detecting method. Another object of the present invention is to provide a manufacturing method and apparatus of a probe-immobilized carrier for use in the detecting method. A further object of the present invention is to provide a recording medium storing data on a manufacturing condition of a probe-immobilized carrier for use in the detecting method, and a kit and system for the detection of a target substance.

A detecting method of a target substance of the present invention is a detecting method of a target substance in a sample, characterized by comprising the steps of:

reacting the sample with a probe-immobilized carrier having an area of a solid phase carrier immobilizing thereon a probe capable of specifically binding to the target substance, and then measuring the presence or absence of binding of the target substance to the probe of the probe-immobilized carrier or the bound amount of the target substance to the probe to obtain a measurement result;

correcting the measurement result on the basis of manufacturing condition data specific to the carrier that influences the measurement result; and

recording the corrected measurement result onto a recording medium or outputting the corrected measurement result.

A manufacturing method of a probe-immobilized carrier according to the present invention is a manufacturing method of a probe-immobilized carrier for the detection of a target substance, storing manufacturing condition data, characterized by comprising the steps of:

forming an area of a solid phase carrier immobilizing a probe thereon; and

recording a forming condition of the area immobilizing the probe thereon that influences a measurement result of the target substance as manufacturing condition data specific to the probe-immobilized carrier manufactured by use of the condition, and storing the manufacturing condition data so that the manufacturing condition data can be read in the detection of the target substance using the probe-immobilized carrier.

A probe-immobilized carrier according to the present invention is a probe-immobilized carrier for the detection of a target substance, storing manufacturing condition data, characterized by comprising: a solid phase carrier; a probe immobilized on the solid phase carrier and being capable of specifically binding to the target substance; and readable data recording a manufacturing condition for the immobilization of the probe onto the solid phase carrier that influences a measurement result of the target substance.

A manufacturing apparatus of a probe-immobilized carrier according to the present invention is a manufacturing apparatus of a probe-immobilized carrier for the detection of a target substance, having carrier holding means for holding a carrier and means for forming an area immobilizing thereon a probe capable of specifically binding to the target substance at a given position of the carrier held by the carrier holding means, characterized by comprising:

manufacturing condition data recording means for recording a forming condition of the area immobilizing the probe thereon that influences a measurement result of the target substance as manufacturing condition data specific to the probe-immobilized carrier.

A measuring apparatus according to the present invention is a measuring apparatus for measuring the presence or absence of binding of a target substance to a probe of a probe-immobilized carrier or the bound amount of the target substance to the probe in reaction between the probe-immobilized carrier and a sample, characterized by comprising:

measuring means for measuring the presence or absence of binding of the target substance to the probe or the bound amount of the target substance to the probe;

reading means for reading manufacturing condition data specific to the probe-immobilized carrier that influences a measurement result obtained with the measuring means;

correcting means for correcting the measurement result of the measuring means on the basis of the manufacturing condition data read with the reading means; and

means for outputting the measurement result corrected with the correcting means.

A medium according to the present invention is a recording medium, characterized by recording manufacturing condition data to be used in a measuring apparatus of the constitution described above.

A kit for the detection of a target substance of the present invention is a kit for the detection of a target substance, characterized by comprising:

a probe-immobilized carrier having a solid phase carrier immobilizing thereon a probe capable of specifically binding to the target substance; and

a recording medium of the constitution described above.

A system for the detection of a target substance of the present invention is a system for the detection of a target substance utilizing a network for the detection of a target substance, characterized by comprising:

a probe-immobilized carrier having a solid phase carrier immobilizing thereon a probe capable of specifically binding to the target substance; and

manufacturing condition data stored in a state capable of being acquired by use of the network and to be used in the measuring apparatus described above.

According to the present invention, it is possible to perform more accurate measurement by recording and storing a manufacturing condition that influences a measurement result of a target substance as manufacturing condition data specific to a probe-immobilized carrier and correcting the measurement result in the detection of the target substance on the basis of this recorded and stored manufacturing condition to eliminate variations in measurement results attributed to the manufacturing condition. According to the present invention, it is possible to provide a detecting method particularly useful for precise measurement using a trace amount of probes.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIG. 1 is a diagram showing the relationship between silane coupling agent concentration and DNA chip performance;

FIG. 2 is a diagram showing the relationship between probe concentration and DNA chip performance;

FIG. 3 is a diagram showing the relationship between the applied amount of a probe and DNA chip performance;

FIG. 4 is a diagram showing the relationship between the applied amount of a probe and the spot area;

FIG. 5 is a diagram showing variations in concentrations among probe solutions;

FIG. 6 is a diagram showing the result of Example 1; and

FIG. 7 is a diagram showing the conceptual illustration of gene diagnosis utilizing a network.

### BEST MODE FOR CARRYING OUT THE INVENTION

Preferred embodiments of the present invention will now be described in detail in accordance with the accompanying drawings.

In the present invention, a condition used in the manufacturing of a probe-immobilized carrier, that is, a condition for immobilizing a probe onto a solid phase carrier, which influences a measurement result in the detection of a target substance, is recorded and stored in a recording medium as manufacturing condition data specific to each probe-immobilized carrier during its manufacturing. When the thus-obtained probe-immobilized carrier storing the manufacturing condition data is used to detect a target substance, it is possible to perform more accurate detection by correcting the measurement result on the basis of the manufacturing condition. For example, even if the concentrations and immobilized amounts of probes vary for handling a trace amount of probes or probe solutions, the variations are measured and stored as manufacturing condition data, and the binding force (bound amount) of the target substance to the probe can be measured strictly by correcting the measurement result on the basis of this manufacturing condition during the detection.

The probe-immobilized carrier according to the present invention comprises a probe enabling the detection of a target substance by specifically binding to the target substance and a solid phase carrier immobilizing this probe thereon, and stores a condition for manufacturing the probe-immobilized carrier (manufacturing condition) that influences a measurement result as readable data.

The solid phase carrier is not particularly limited as long as it immobilizes the probe thereon and provides for the detection or separation of a target substance without any trouble by use of the obtained probe-immobilized carrier. To take a microarray as an example, the solid phase carrier is preferably a glass substrate or plastic substrate, particularly preferably an alkali-free glass substrate or quartz substrate without alkali components, in consideration of the detection of a target substance and general versatility. Various methods are known as methods of immobilizing a probe onto a solid phase carrier. To take DNA as an example, an immobilizing method thereof includes a method of immobilizing a probe onto a substrate by synthesizing the probe on the substrate and a method of immobilizing a probe onto a substrate by applying a probe prepared in advance onto a substrate by a pin, stamp, or ink-jet method.

The immobilizing method performed by synthesizing a probe on a solid phase carrier is disclosed in, for example U.S. Pat. No. 5,143,854. In the method disclosed therein, a protecting group is removed from a selected area of a solid phase carrier by use of an activator, and the binding of a monomer with a removable protecting group to the area is repeated to synthesize polymers of varying lengths on the solid phase carrier. Alternatively, the immobilizing method performed by applying a probe prepared in advance onto a substrate is disclosed in, for example Japanese Patent Laid-Open No. H08-23975. In the method disclosed therein, a material for immobilization consisting of a substrate and a high molecular compound with a carbodiimide group held on the substrate is contacted with a biologically active substance having reactivity with carbodiimide group to immobilize the biologically active substance. As described in Japanese Patent Laid-Open No. H08-334509, a method of detecting a biologically active substance by using the immobilization of the biologically active substance via a carbodiimide group onto a compound having carbodiimide is known. Furthermore, Japanese Patent Laid-Open No. 2001-178442 has disclosed that a DNA fragment having a thiol group at the terminal portion is contacted in a liquid phase with a solid phase carrier having surface on which a chain molecule having a reactive substituent capable of reacting with a thiol group and forming covalent bond is immobilized via one end thereof. In the method described therein, the DNA fragment is immobilized onto the surface of the solid phase carrier by forming the covalent bond between the DNA fragment and the chain molecule, and the reactive substituent capable of reacting with a thiol group and forming covalent bond is concretely a substituent containing a group selected from the group consisting of maleimidyl, α,β-unsaturated carbonyl, α-halocarbonyl, halogenated alkyl, aziridine and disulfide groups.

Many immobilizing methods of a probe are known, as to immobilizing methods of a DNA fragment even alone, as described above. In the present invention, the type or immobilization mechanism of a probe is not particularly limited.

A method of spotting an aqueous solution of a probe dissolved or dispersed in an aqueous medium onto a substrate with a reactive group by an ink-jet or pin method as disclosed in Japanese Patent Laid-Open No. H11-187900 is known as a method of applying a probe onto a substrate. Among the spotting methods described above, particularly the ink-jet method is a preferable spotting method because it can achieve accurate spotting at high speed and high density. In the ink-jet method, a probe-containing solvent is placed in a very thin nozzle, and a trace amount of the probe-containing solvent is allowed to accurately jump out of the tip (ejection port) of the nozzle by instantly pressurizing or heating the neighborhood of the tip of the nozzle. Thus, the probe-containing solvent is allowed to fly in the air and attached to substrate surface. The spotting method performed by the ink-jet method uses a component contained in the probe solution that does not substantially influence the probe ejected as the probe solution from the ink-jet head. The component is not particularly limited as long as it satisfies medium composition normally ejectable onto the substrate by use of the ink-jet head. For example, when the ink-jet head is a bubble-jet head provided with a mechanism to eject a medium by applying thermal energy thereto, a liquid containing glycerin, thiodiglycol, isopropyl alcohol and acetylene alcohol is preferable as the component contained in the probe solution. To be more specific, a liquid containing 5 to 10% by weight of glycerin, 5 to 10% by weight of thiodiglycol and 0.5 to 1% by weight of acetylene alcohol is preferably used as the probe solution. Alternatively, when the ink-jet head is a piezo-jet head that ejects a medium by use of a piezoelectric element, a liquid containing ethylene glycol and isopropyl alcohol is preferable as the component contained in the probe solution. To be more specific, a liquid containing 5 to 10% by weight of ethylene glycol and 0.5 to 2% by weight of isopropyl alcohol is preferably used as the probe solution.

When the probe solution thus obtained is ejected from the ink-jet head and attached to the substrate, spots are circular in shape and are ejected within a range that is not spread out. Even when the probe solution is spotted at high density, the linkage between adjacent spots can be suppressed effectively.

When an attempt is made to accurately measure the binding force (bound amount) of a target substance to a probe, it is desirable that each probe should be the same in shape and be bound in the same amount. From this point of view, the ink-jet system is a very preferable spotting method.

The probe utilized in the present invention is any of those capable of being immobilized on the solid phase carrier and specifically binding to a target substance. Various substances are known to be available as probes. For example, nucleic acids (e.g., oligonucleotide and DNA fragments), proteins or peptides typified by antibodies and enzymes, and also saccharides can be used. The probe-immobilized carrier may have the solid phase carrier on which plural areas immobilizing the probe thereon (which refer to areas distinguishable from areas not immobilizing the probe thereon) are formed. Moreover, the probe-immobilized carrier may have areas immobilizing plural types of probes thereon.

DNA chips using DNA as a probe are broadly classified into those using oligonucleotide as a probe or those using cDNA as a probe. In both cases, the probes are often expensive or valuable, and the amount of probes or probe solution are desired to be reduced, as described above. However, it is often difficult to accurately handle a trace amount of droplets. According to the present invention, even if manufacturing conditions such as probe concentrations that influence performance vary, it is possible to perform accurate measurement by measuring in advance the variations or analyzing in advance the amount of the probes and correcting the result with an analyzing apparatus.

One of the conditions that influence a measurement result includes a reaction time between the probe and the solid phase carrier. The reaction time required for the binding reaction between the probe and the solid phase carrier is several minutes to several hours, though differing depending on immobilization mechanisms, temperatures, and so on. It is difficult to simultaneously spot many types of probes. Therefore, in general, one to several types of probes are simultaneously spotted, and this procedure is appropriately repeated. However, a time required for immobilization varies between the probe spotted first and the probe spotted last. The reaction efficiency between the probe and the solid phase carrier is changed with such change in binding reaction time. Since the ink-jet method completes spotting at high speed as described above, the influence of such time difference is small. However, particularly the pin method is generally time-consuming in most cases when a large number of chips are simultaneously manufactured. Therefore, a more accurate result can be obtained by recording a spotting time (actual binding reaction time) and outputting an analysis result in consideration of this time with an analyzing apparatus.

Additional conditions include the amount (density) of the probe immobilized on the solid phase carrier. Conditions that specify this immobilized amount of the probe include the followings:

a probe concentration of the probe solution applied to the solid phase carrier;

the amount (density) of an immobilizing agent on the surface of the solid phase carrier when the probe is immobilized by arranging the immobilizing agent for probe immobilization onto the surface of the solid phase carrier and reacting this immobilizing agent with the probe solution; and

an immobilizing agent concentration of the immobilizing agent solution applied to the solid phase carrier.

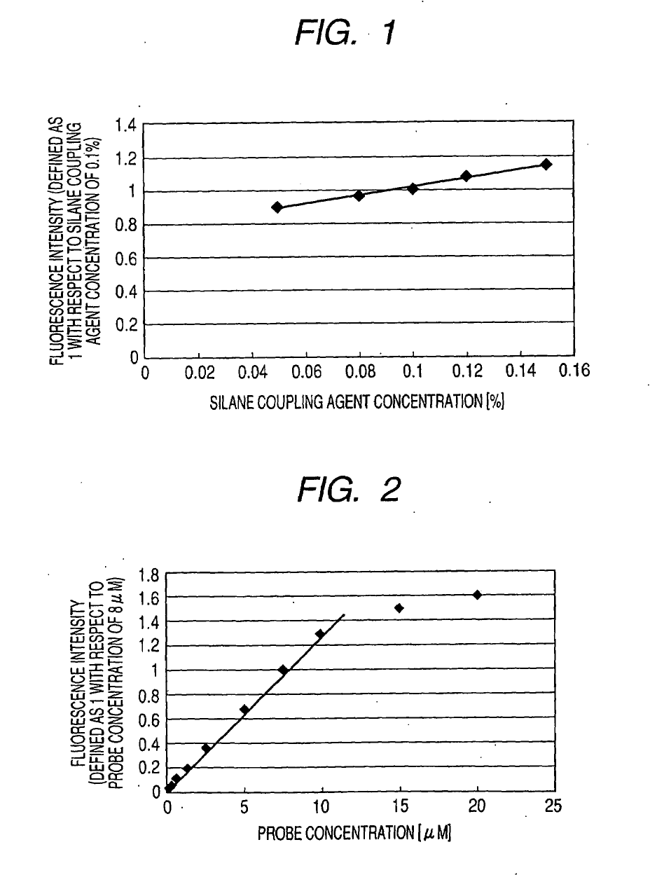

For example, in Examples disclosed in Japanese Patent Laid-Open No. H11-187900, a washed glass substrate is treated with 1 wt % aqueous solution of an aminosilane coupling agent and 0.3 mg/mL N-(6-maleimidocaproyloxy)succinimide solution as immobilizing agent solutions. Particularly with change in the concentration of this silane coupling agent among these two types of immobilizing agents, DNA chip performance is also changed (see FIG. 1, which illustrates fluorescence intensity defined as 1 with respect to a silane coupling agent concentration of 0.1 wt % in the reaction between a probe solution with a constant probe concentration and a target substance solution containing a target substance in an sufficient amount exceeding an amount required for the reaction). Next, difference in DNA chip performance among varying probe concentrations of probe solutions is shown in FIG. 2 (which illustrates fluorescence intensity defined as 1 with respect to a probe concentration of 8 μM). As the probe concentration is increased and exceeds a certain level, the immobilized amount of the probe naturally becomes constant. However, such concentrations require using a considerable amount of expensive probes and are disadvantageous in terms of cost. Furthermore, unreacted probes, when removed, are bound outside of the spots, leading to a rise in background. Therefore, it is generally desirable that the probe should be used at a concentration that is not so high. Thus, an area (e.g., an area with a probe concentration of 10 μM or less in the system shown in FIG. 2) in which the probe concentration and the DNA chip performance are in almost proportional relationship with each other is often used.

From these points of view, it is evidently useful to record, as the manufacturing conditions, not only the concentration of a probe solution but also the concentrations of all materials necessary to manufacture the probe-immobilized carrier, and give feedback on the manufacturing conditions to an analyzing apparatus. For example, when the probe is immobilized onto the solid phase carrier by use of an immobilizing agent, it is preferred that an immobilizing agent concentration of an immobilizing agent solution used in the treatment of the surface of the solid phase carrier and a probe concentration of a probe solution should be recorded as the manufacturing conditions and used in the correction of a measurement result.

The immobilizing agent concentration of the immobilizing agent solution can be measured by a variety of methods known in the art. For example, atomic absorption, the refractive index of the solution or an automatic titration apparatus is selected according to the type of the immobilizing agent. Since the limited type of the immobilizing agent is used in the manufacturing of the probe-immobilized carrier, a measuring method necessary for the concentration measurement is easily selected, and the concentration measurement can be performed easily.

However, a probe-immobilized carrier generally called a microarray immobilizes several types to several tens of thousands of types of probes thereon according to its use and so on. In consideration of a large number of probes measured, a preferable method of measuring the concentration of the probe solution is to measure the concentration of the probe solution in an applicator that applies the probe solution onto the solid phase carrier or in a dispensing apparatus that supplies the probe solution retained in the applicator. The applicator of the probe solution that can be utilized is a probe solution applicator comprising an ejection port for ejecting a liquid, a reservoir accommodating the probe solution, and a liquid path connecting the ejection port and the reservoir. The probe concentration is measured at a position in this applicator capable of measuring the probe concentration.

For example, Japanese Patent Laid-Open No. 2003-63013 has disclosed an ink-jet head incorporating a light-emitting element and a light-receiving element therein. To take DNA such as oligonucleotide or cDNA as an example of a probe, a concentration is calculated from absorbance obtained by emitting light with a wavelength of 260 nm from the light-emitting element and receiving the light by the light-receiving element. As a result, it is possible to automatically measure the concentrations of plural types of probe solutions in a short time. The conversion of the absorbance to the concentration must be calculated in advance because molar extinction coefficients are different among probes.

It is also possible to measure the concentration in a multi-well plate accommodating plural probe solutions or in the process of dispensing the solutions to the multi-well plate or the like. A manufacturing apparatus described in the present invention also contains such a dispenser. In this context, a measuring method is not particularly limited as long as it can measure the concentration of the probe solution or other materials.

Although such a method is capable of measuring the concentrations of many probes, the creation of a calibration curve for each of tens of thousands of types of probes takes much time. In such a case, a method of creating a calibration curve by simulation can also be used. For example, with increases in probe length, reaction efficiency with the solid phase carrier is expected to be reduced due to the influence of steric hindrance. Therefore, it is preferred that the length and possible three-dimensional structure of the probe should be used as parameters to perform the simulation of the influence thereof on the concentration and reaction efficiency, which is in turn used as a calibration curve.

However, to take a DNA chip as an example, which usually immobilizes many probes thereon, it is preferred that the melting temperatures (Tm) of the probes should be set to almost equal values. Therefore, the lengths of the probes are almost equal. From this point of view, the creation of all calibration curves by actual measurement or simulation is preferable. However, it is also possible to apply the calibration curves of one to several types of probes to other probes.

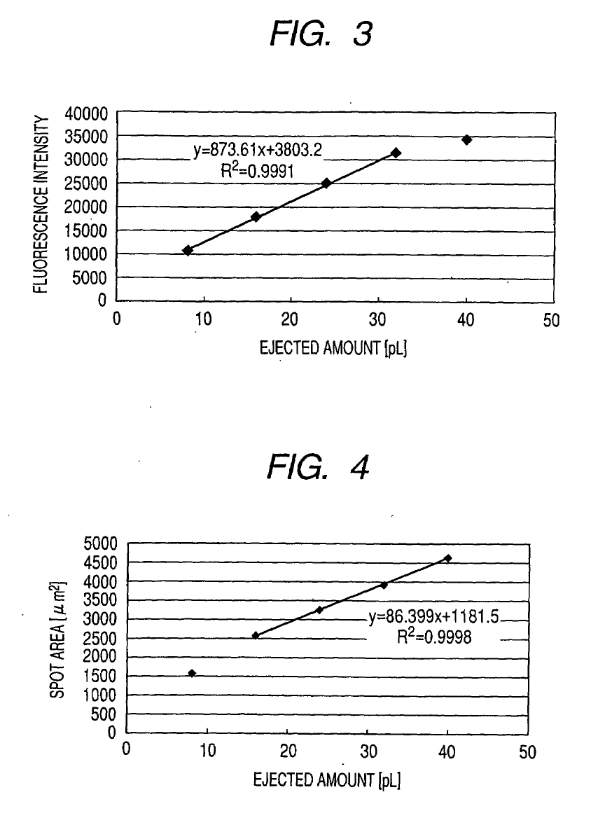

A factor that specifies the immobilized amount of the probe further includes the applied amount of the probe solution (see FIG. 3, which measures fluorescence intensity in the reaction between a probe solution with a constant probe concentration and a target substance solution containing a target substance in an sufficient amount exceeding an amount required for the reaction). In the system practiced this time, an ejected amount and fluorescence intensity are in almost proportional relationship with each other as long as the ejected amount is approximately 16 pL or less.

When the probe is spotted by the pin method, the shape and applied amount of the probe are not constant in most cases. Therefore, it is effective to measure, after spotting, the volume of the spot by use of a confocal laser microscope or the like and give feedback on the volume to an analyzing apparatus. However, when the probe is spotted by the ink-jet system, each spot is applied in a uniform amount. Therefore, the volume can be calculated from the area if the contact angles thereof on a substrate are equal. Thus, it is also possible to use a measuring apparatus capable of measuring only two dimensions. FIG. 4 shows the relationship between an ejected amount and a spot area. In the system used this time, the spot area and the ejected amount are shown to be in proportional relationship with each other within a range from approximately 8 pL to approximately 20 pL. In this range, the applied amount of the probe solution can be calculated from the area without measuring the volume of the spot.

In brief, the manufacturing condition specific to the probe-immobilized carrier that can be used in the present invention is at least one of

(1) at least either an area or a shape of the area immobilizing the probe thereon,
(2) a value obtained by measuring the amount of the probe immobilized on the probe-immobilized carrier, and
(3) an immobilizing agent concentration of an immobilizing agent solution when the probe is immobilized by treating the surface of the solid phase carrier with the immobilizing agent solution. When the area immobilizing the probe thereon is formed by applying the probe solution to the solid phase carrier, the manufacturing condition can be selected from among
(A) a probe concentration of the probe solution,
(B) absorbance of the probe solution,
(C) a reaction time of reaction for binding the probe contained in the probe solution to the solid phase carrier, and
(D) a measured value of at least any applied volume of the probe solution,
in addition to the conditions (1) to (3).

The manufacturing conditions described above are merely manufacturing conditions and do not directly reflect the immobilized amount of the probe. These manufacturing conditions indirectly provide data on the immobilized amount (density) of the probe onto the solid phase carrier and can be measured by a simple method. However, the further accurate immobilized amount (density) of the probe is required depending on the type of the probe, the intended use of the probe-immobilized carrier, etc. In such a case, it is preferred that the amount (density) of the probe immobilized on the solid phase carrier should be measured directly and recorded as the manufacturing condition.

A method of measuring probe density is disclosed in Japanese Patent Laid-Open No. 2004-85546. In this disclosure, TOF-SIMS (time-of-flight secondary ion mass spectrometry) is used to analyze a DNA chip. While this method serves as a destructive inspection, that is, a sampling inspection, it allows for two-dimensional imaging with excellent quantitative properties of the DNA chip and further for composition analysis and is therefore preferable as an analyzing method of the present invention. The analyzing method is not limited to these examples. The manufacturing apparatus described in the present invention also encompasses those not physically incorporating therein an apparatus such as the TOF-SIMS that analyzes the manufacturing conditions such as the amount (density) of the probe.

In the present invention, the manufacturing condition that influences a measurement result is applied to the probe-immobilized carrier as data specific to the probe-immobilized carrier. The record about the manufacturing condition applied to each probe-immobilized carrier is stored in a variety of recording media or in memory in a server in a network and is kept readable as data for the correction of a measurement result in the detection of a target substance using the probe-immobilized carrier.

The recording medium may be installed in the probe-immobilized carrier or included in the same package with the probe-immobilized carrier. For example, when data size is small as in the immobilizing agent solution concentration (e.g., the silane coupling agent concentration), a method of recording the data in a bar code or two-dimensional code form onto a portion of the probe-immobilized carrier not immobilizing the probe thereon can be used. However, when data size is large as in the probe solution concentration or probe-immobilized amount of a bio chip (e.g., the DNA chip) having a large number of probes, the bar code or two-dimensional code has a limitation on recordable data size. From this point of view, a method may be adopted, in which the data is recorded onto a recording medium with large storage capacity such as IC tag, and this recording medium is attached to the probe-immobilized carrier itself or integrated into a case that supports the probe-immobilized carrier. Another possible method is to record the data onto a recording medium such as an optical disk (e.g., CD-ROM), a magnetic disk, a magneto-optical disk and flash memory and provide, to a user, this recording medium included in the same package with the probe-immobilized carrier.

In a further alternative method, the recording medium with the record of the manufacturing condition data or electronic data in the server in a network is stored by the manufacturer of the probe-immobilized carrier, and a computer storing this data is connected via a network to an analyzing apparatus of an user described below. In this case, when the user employs the probe-immobilized carrier, the manufacturing condition is downloaded and used. In this case, it is preferred that an identifier for the probe-immobilized carrier such as a serial number or lot number should be attached to the probe-immobilized carrier, the case supporting it, or the package. In a preferable method, the user may send this identifier to a computer possessed by the manufacturer and download and read the manufacturing condition of the probe-immobilized carrier corresponding to the identifier.

The type of the recording medium and the method of providing the data to the analyzing apparatus are not limited to these examples.

To take a probe concentration as an example, probe-immobilized carriers are produced in advance under conditions of varying probe concentrations in order to perform correction. The probe of the probe-immobilized carrier is hybridized with a target substance labeled with, for example a fluorescent material and washed, followed by detection with a fluorescence detector. This concentration and the result from the fluorescence detector are made into a graph (see FIG. 2). In this procedure, even if the same probe-immobilized carrier is used, a detection result differs depending on user atmospheres (e.g., target substance concentrations, hybridization temperatures and washing conditions). From this point of view, it is preferred that a calibration curve should be normalized not based on the measurement result but by determining a certain criterion and using a detection result under this criterion as a reference value (in FIG. 2, fluorescence intensity is defined as 1 with respect to a probe concentration of 8 μM used as a reference).

To take a DNA chip as an example, Tm differs depending on probe sequences. Therefore, it is preferred that a calibration curve should be created for not one representative probe but all the probes. For a DNA chip designed to immobilize thereon probes with almost equal Tm values, the calibration curves of one or several types of probes may be applied to other probes, as described above. The algorithm for performing the correction is not limited to these examples.

One or two or more manufacturing conditions useful for the correction of a measurement result can be selected from the manufacturing conditions described above and recorded to correct the measurement result.

There are various types of analyzing apparatuses that react the probe of the probe-immobilized carrier thus manufactured with a target substance to measure the bound amount of the target substance. To take a DNA chip as an example, the method as described above is often used, in which a target substance labeled in advance with a fluorescent material is hybridized with the probe of the probe-immobilized carrier and washed, followed by detection with a fluorescence detector. In this case, if the bound amount of each probe differs, the amount or binding force of the target substance cannot be measured accurately. Therefore, the bound amount is read from the recording medium with the record of the amount of each probe of the probe-immobilized carrier to correct the measurement result by use of the calibration curve.

Variations in the measurement results thus corrected are significantly reduced even if the manufacturing conditions, for example probe concentrations, vary.

The analyzing apparatus used in the present invention is not limited to the fluorescence detector and can also adopt the method as disclosed in Japanese Patent Laid-Open No. 05-199898. In this method, a nucleic acid probe immobilized on the surface of an electrode is used, and an electrochemically active double strand recognizing body capable of specifically binding to a double-stranded nucleic acid is added to a reaction system between the nucleic acid probe and a gene sample. In the method, the double strand recognizing body bound with the double-stranded nucleic acid composed of the nucleic acid probe and the gene of interest is detected by electrochemical measurement via the electrode, thereby detecting the presence of the nucleic acid probe hybridized with the gene of interest. This method can also be utilized in the present invention.

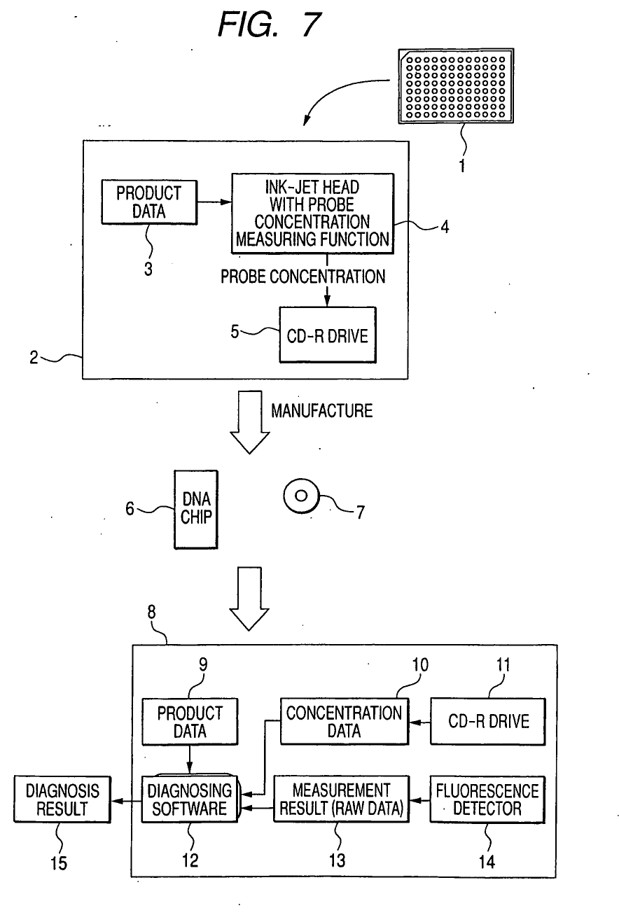

An example of a gene diagnosis system of the present invention is shown in FIG. 7. A manufacturer possesses a probe spotter 2. This spotter measures the concentration of a probe solution 1 by use of an ink-jet 4 with probe concentration measuring function described above and applies and immobilizes the probe solution onto a solid phase carrier to produce a DNA chip 6. In this procedure, product data 3 involving an instruction that specifies which probe is applied to which position is used to determine an applied position. The measured concentration is recorded as each probe concentration onto a recording medium 7 such as CD-R by use of a recording apparatus 5 such as a CD-R drive. The DNA chip and the medium with the record of concentration data are provided to a user.

The user hybridizes the probe on the DNA chip to a target substance and measures this hybridization with a fluorescence detector 14 of an inspecting apparatus 8 to obtain raw data 13 as the measurement result. Moreover, the medium with the record of concentration data is loaded into a drive 11 of an analyzing apparatus. A diagnosis result 15 is drawn with diagnosing software 12 from the concentration data 10 recorded in the CD-R, a value from raw data of the measurement result corrected with the concentration data, and product data 9 about which probe is arranged at which position on the DNA chip.

Furthermore, at least a probe-immobilized carrier of the constitution described above and a recording medium of the constitution described above recording a manufacturing condition can be used to constitute a kit for the detection of a target substance. This kit can further be supplemented with a reagent for reacting the probe with the target substance, a reagent for detecting the formed hybrid, etc. As described above, the manufacturing condition data may be recorded onto an appropriate area of the probe-immobilized carrier used as the recording medium. Alternatively, the manufacturing condition data may be written into a recording medium prepared separately, which is in turn included apart from the probe-immobilized carrier but in the same package or immobilized onto the probe-immobilized carrier.

### Example

Hereinafter, the present invention will be described more in detail with reference to Example.

### Example 1

A correcting method of fluorescence intensity for variations in probe concentrations will be described.

### (1) Production of Substrate

A glass substrate (slide glass) was immersed for 10 minutes in 1 mol/l aqueous solution of sodium hydroxide heated in advance to 60° C. Subsequently, the slide glass was well rinsed in running pure water to wash off the sodium hydroxide attached to the slide glass. After sufficient rinsing, the slide glass was immersed in pure water and ultrasonically cleaned for 10 minutes. After ultrasonic cleaning, the slide glass was well rinsed in running pure water to wash off particles attached to the slide glass. Then, this slide glass was spin-dried. An aminosilane coupling agent (trade name: KBM-603; manufactured by Shin-Etsu Chemical Co., Ltd.) was dissolved at a concentration of 1% by weight and stirred for 30 minutes. The slide glass was immersed for 30 minutes in this aqueous solution, then taken out, and washed with water. The slide glass was placed in an oven and dried at 120° C. for 1 hour.

### (2) Synthesis of Probe

In this Example, a probe used was a single-stranded nucleic acid having a nucleotide sequence complementary to a partial or entire nucleotide sequence of a target nucleic acid to be detected and specifically hybridizing to the nucleotide sequence of the target nucleic acid under stringent conditions illustrated by conditions below. A single-stranded nucleic acid of SEQ ID NO: 1 was synthesized with an automatic DNA synthesizer. A mercapto group was introduced into the terminus of the single-stranded DNA of SEQ ID NO: 1 by using Thiol-Modifier (manufactured by Glen Research) during its synthesis with the automatic DNA synthesizer. Subsequently, the single-stranded DNA was subjected to typical deprotection and collected. The DNA was purified by high-performance liquid chromatography and used in experiments below.

### 5′HS—(CH₂)₆—O—PO₂—O-ACTGGCCGTCGTTTTACA3′ (SEQ ID NO: 1)

### (3) Immobilization of Probe

At first, an aqueous solution containing 7.5% by weight of glycerin, 7.5% by weight of urea, 7.5% by weight of thiodiglycol and 1% by weight of acetylene alcohol (trade name: Acetylenol E100; manufactured by Kawaken Fine Chemicals Co., Ltd.) was prepared.

Two types of DNA fragments (SEQ ID NO: 1) synthesized in the paragraph (2) were separately dissolved at a concentration of 0.156 μM, 0.313 μM, 0.626 μM, 1.25 μM, 2.5 M, 5 μM, 8 μM, 10 μM or 20 M in the aqueous solution. Each of these aqueous solutions containing the DNA fragment was separately spotted with a bubble-jet printer onto the slide glass produced by the method of the paragraph (1). In observation with a 15-magnification loupe, no satellite spot (spot derived from a spray from a liquid landing on solid phase surface) was observed. The slide glass with the spots of the solution containing the probe was left at room temperature for 10 minutes and washed with 1 M NaCl/50 mM phosphate buffer solution (pH 7.0).

### (4) Blocking and Hybridization Reaction

Bovine serum albumin was dissolved at a concentration of 1.0% by weight in 1 M NaCl/50 mM phosphate buffer solution (pH 7.0). The DNA chip produced by the method described above was immersed at room temperature for 2 hours in this solution to perform blocking reaction. Rhodamine was bound to the 5′ terminus of a DNA fragment having a nucleic acid sequence complementary to that of the probe of SEQ ID NO: 1 to synthesize a labeled DNA fragment. This labeled DNA fragment was dissolved at a concentration of 50 nM in 1 M NaCl/50 mM phosphate buffer solution (pH 7.0). The blocked DNA chip was immersed in this solution containing the labeled DNA fragment and left at 45° C. for 2 hours. Then, the unreacted DNA fragment was washed off with 1 M NaCl/50 mM phosphate buffer solution (pH 7.0). The DNA chip was further washed with pure water.

### (5) Relationship Between Concentration and Fluorescence Intensity

The hybridized DNA chip was fluorescently observed at a wavelength of 532 nm with a fluorescence scanner (trade name: GenePix 4000B; manufactured by Axon Instruments, Inc). As a result, each spot was almost circular with a diameter of 45 μm. The fluorescence intensity was measured at PMT of 400 V and laser power of 100% and defined as 1 with respect to a probe concentration of 8 μM to determine the ratio thereof. The ratio is shown in a graph form in FIG. 2. As can be seen from FIG. 2, the probe concentration and the fluorescence intensity in this system are in proportional relationship with each other at a concentration of 10 μM or less, as described above.

### (6) Production of DNA Chip

Oligonucleotide of SEQ ID NO:1 was dispensed in advance at a concentration of 0.0125 nmol to 24 microtubes with an automatic dispensing system and then dried. This was dissolved (at a concentration of 8 μM on calculation) in 100 mL of aqueous solution containing 7.5% by weight of glycerin, 7.5% by weight of urea, 7.5% by weight of thiodiglycol and 1% by weight of acetylene alcohol (trade name: Acetylenol E100; manufactured by Kawaken Fine Chemicals Co., Ltd).

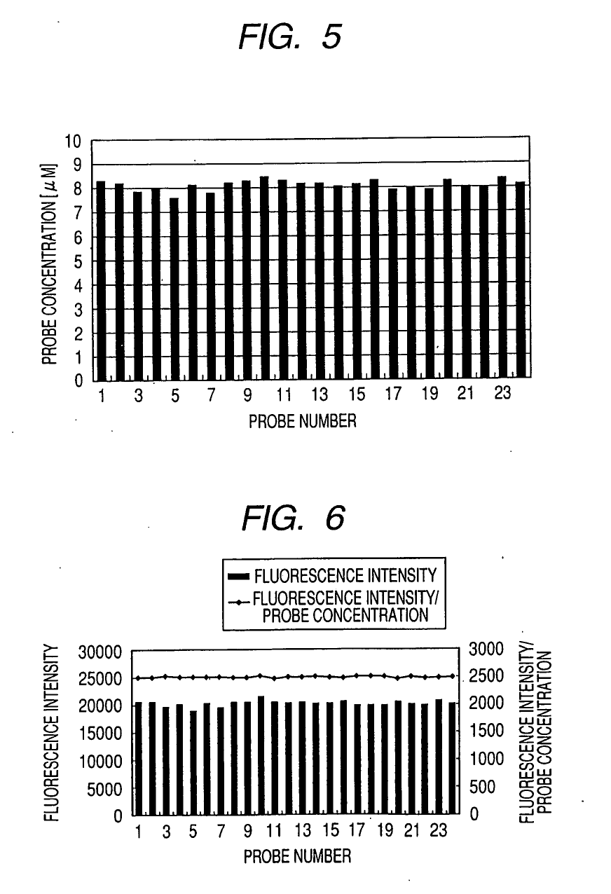

This solution was in turn placed into a 96-well multi-well plate. The absorbance of 24 oligonucleotides was measured with a plate reader and converted to a concentration. This result is shown in FIG. 5. In this measurement, the 3σ/average absorbance of 24 oligonucleotides was 0.078. Each of the probe solutions was transferred from this well plate to a reservoir of the bubble-jet head and separately spotted onto the slide glass produced in the paragraph (1). The slide glass with the spots of the solution containing the probe was left at room temperature for 10 minutes and washed with 1 M NaCl/50 mM phosphate buffer solution (pH 7.0).

### (7) Blocking and Hybridization Reaction

Blocking and hybridization reaction were performed in the same way as in the paragraph (4).

### (8) Result

The hybridized DNA chip was fluorescently observed at a wavelength of 532 nm with a fluorescence scanner (trade name: GenePix 4000B; manufactured by Axon Instruments, Inc). As a result, each spot was almost circular with a diameter of 45 μm. The fluorescence intensity was measured at PMT of 400 V and laser power of 100%. The result is shown in FIG. 6. The 3σ/average fluorescence intensity of these 24 spots was 0.066. As shown in the paragraph (5), the probe concentration and the fluorescence intensity in the concentration area used this time were in proportional relationship with each other. This fluorescence intensity was corrected by dividing it by the concentration shown in FIG. 5. The result is shown in FIG. 6. The 3σ/average fluorescence intensity after this correction was 0.016.

As described above, variations are reduced more drastically in the corrected result than in the result not corrected.

As describe above, it is possible to spot one type of probe onto plural areas and correct measurement results fluctuated due to variations in concentrations thereof. Even when plural types of probes are spotted onto an identical solid phase carrier, it is possible to correct measurement results by performing similar procedures on each of the probes.

The manufacturing of a probe-immobilized carrier having a solid phase carrier immobilizing expensive and valuable probes thereon requires a technique of handling a small amount of probes or probe solutions. The accurate handling of a small amount has a limitation. However, these results have shown that the use of the present invention produces an accurate detection result even with a lack of a part of accuracy.

The present invention is not limited to the above embodiments and various changes and modifications can be made within the spirit and scope of the present invention. Therefore to apprise the public of the scope of the present invention, the following claims are made.

This application claims priority from Japanese Patent Application No. 2005-191697 filed Jun. 30, 2005, which is hereby incorporated by reference herein.

1. A detecting method of a target substance in a sample, characterized by comprising the steps of:
reacting the sample with a probe-immobilized carrier having an area of a solid phase carrier immobilizing thereon a probe capable of specifically binding to the target substance, and then measuring the presence or absence of binding of the target substance to the probe of the probe-immobilized carrier or the bound amount of the target substance to the probe to obtain a measurement result;
correcting the measurement result on the basis of manufacturing condition data specific to the carrier that influences the measurement result; and
recording the corrected measurement result onto a recording medium or outputting the corrected measurement result.

2. The detecting method according to claim 1, wherein the corrected measurement result is obtained by creating in advance a calibration curve of the amount of the target substance measured from reaction between a probe-immobilized carrier manufactured under a changed manufacturing condition and the target substance, and correcting the measurement result by use of the calibration curve.

3. The detecting method according to claim 1, wherein the corrected measurement result is obtained by simulating in advance how the amount of the target substance binding to the probe changes with change in a manufacturing condition of the probe-immobilized carrier, and correcting the measurement result by use of the simulation result.

4. The detecting method according to claim 1, wherein plural types of probes are immobilized on the probe-immobilized carrier.

5. The detecting method according to claim 1, wherein the manufacturing condition data involves at least one of (1) at least either an area or a shape of the area immobilizing the probe thereon, (2) a value obtained by measuring the amount of the probe immobilized on the probe-immobilized carrier, and (3) an immobilizing agent concentration of an immobilizing agent solution when the probe is immobilized by treating the surface of the solid phase carrier with the immobilizing agent solution.

6. The detecting method according to claim 1, wherein the probe-immobilized carrier is manufactured by applying a probe solution containing the probe to the solid phase carrier, and the manufacturing condition data involves a value obtained by measuring at least any of (1) a probe concentration of the probe solution, (2) absorbance of the probe solution, (3) a reaction time of reaction for binding the probe contained in the probe solution to the solid phase carrier, and (4) a volume of the probe solution applied.

7. The detecting method according to claim 6, wherein the probe solution is applied to the solid phase carrier with a probe solution applicator comprising an ejection port for ejecting a liquid, a reservoir accommodating the probe solution, and a liquid path connecting the ejection port and the reservoir, and the absorbance is measured in the probe applicator.

8. The detecting method according to claim 1, wherein the probe is a nucleic acid.

9. A manufacturing method of a probe-immobilized carrier for the detection of a target substance, storing manufacturing condition data, characterized by comprising the steps of:
forming an area of a solid phase carrier immobilizing a probe thereon; and
recording a forming condition of the area immobilizing the probe thereon that influences a measurement result of the target substance as manufacturing condition data specific to the probe-immobilized carrier manufactured by use of the condition, and storing the manufacturing condition data so that the manufacturing condition data can be read in the detection of the target substance using the probe-immobilized carrier.

10. The manufacturing method according to claim 9, wherein plural types of probes are immobilized on the solid phase carrier.

11. The manufacturing method according to claim 9, wherein the manufacturing condition data involves at least one of (1) at least either an area or a shape of the area immobilizing the probe thereon, (2) a value obtained by measuring the amount of the probe immobilized on the probe-immobilized carrier, and (3) an immobilizing agent concentration of an immobilizing agent solution when the probe is immobilized by treating the surface of the solid phase carrier with the immobilizing agent solution.

12. The manufacturing method according to claim 9, wherein the probe-immobilized carrier is manufactured by applying a probe solution containing the probe to the solid phase carrier, and the manufacturing condition data involves a value obtained by measuring at least one of (1) a probe concentration of the probe solution, (2) absorbance of the probe solution, (3) a reaction time of reaction for binding the probe contained in the probe solution to the solid phase carrier, and (4) a volume of the probe solution applied.

13. The manufacturing method according to claim 12, wherein the application of the probe solution to the solid phase carrier is performed with a probe solution applicator comprising an ejection port for ejecting a liquid, a reservoir accommodating the probe solution, and a liquid path connecting the ejection port and the reservoir, and the absorbance is measured in the probe applicator.

14. The manufacturing method according to claim 9, wherein the probe is a nucleic acid.

15. A probe-immobilized carrier for the detection of a target substance, storing manufacturing condition data, characterized by comprising: a solid phase carrier; a probe immobilized on the solid phase carrier and being capable of specifically binding to the target substance; and readable data recording a manufacturing condition for the immobilization of the probe onto the solid phase carrier that influences a measurement result of the target substance.

16. A manufacturing apparatus of a probe-immobilized carrier for the detection of a target substance, having carrier holding means for holding a carrier and means for forming an area immobilizing thereon a probe capable of specifically binding to the target substance at a given position of the carrier held by the carrier holding means, characterized by comprising:
manufacturing condition data recording means for recording a forming condition of the area immobilizing the probe thereon that influences a measurement result of the target substance as manufacturing condition data specific to the probe-immobilized carrier.

17. A measuring apparatus for measuring the presence or absence of binding of a target substance to a probe of a probe-immobilized carrier or the bound amount of the target substance to the probe in reaction between the probe-immobilized carrier and a sample, characterized by comprising:
measuring means for measuring the presence or absence of binding of the target substance to the probe or the bound amount of the target substance to the probe;
reading means for reading manufacturing condition data specific to the probe-immobilized carrier that influences a measurement result obtained with the measuring means;
correcting means for correcting the measurement result of the measuring means on the basis of the manufacturing condition data read with the reading means; and
means for outputting the measurement result corrected with the correcting means.

18. The measuring apparatus according to claim 17, wherein the measuring apparatus stores data of a calibration curve of the amount of the target substance measured from reaction between a probe-immobilized carrier manufactured under a changed manufacturing condition and the target substance, and the correcting means is means for performing the correction by use of the calibration curve.

19. The measuring apparatus according to claim 17, wherein the measuring apparatus stores a result of simulating in advance how the amount of the target substance binding to the probe changes with change in a manufacturing condition of the probe-immobilized carrier, and the correcting means is means for performing the correction by use of the simulation result.

20. A recording medium, characterized by recording manufacturing condition data to be used in a measuring apparatus according to claim 17.

21. A kit for the detection of a target substance, characterized by comprising:
a probe-immobilized carrier having a solid phase carrier immobilizing thereon a probe capable of specifically binding to the target substance; and
a recording medium according to claim 20.

22. The kit according to claim 21, wherein the recording medium is arranged in an area of the solid phase carrier not immobilizing thereon the probe.

23. A system for the detection of a target substance utilizing a network for the detection of a target substance, characterized by comprising:
a probe-immobilized carrier having a solid phase carrier immobilizing thereon a probe capable of specifically binding to the target substance; and
manufacturing condition data stored in a state capable of being acquired by use of the network and to be used in a measuring apparatus according to claim 17.


SEQUENCE LISTING (1 sequence)

SEQ ID NO: 1 - DNA, 18 bp, Artificial
  Probe for detection
actggccgtcgttttaca